(19) United States
(12) Patent Application Publication
(10) Pub. No.: US 2021/0100524 A1
(43) Pub. Date: Apr. 8, 2021

(54) Vector flow estimation in medical ultrasound using deep learning neural networks

(71) Applicant: The Board of Trustees of the Leland Stanford Junior University, Stanford, CA (US)

(72) Inventors: You Li, Stanford, CA (US); Jeremy J. Dahl, Palo Alto, CA (US)

(21) Appl. No.: 17/064,270

(22) Filed: Oct. 6, 2020

Related U.S. Application Data
(60) Provisional application No. 62/911,602, filed on Oct. 7, 2019.

(51) Int. Cl. A61B 8/06; A61B 8/08; G06N 3/04

(52) U.S. Cl. CPC A61B 8/06; G06N 3/04; A61B 8/488

(57) ABSTRACT

A method and system are provided to characterize blood flow dynamics in vivo. A Doppler acquisition is obtained in vivo with two or more transmit-receive event pairs at different angles from which three-dimensional angle-resolved RF data is determined. Four-dimensional dimensional angle-resolved RF data is obtained by repeating these steps. The four-dimensional angle-resolved RF data is then provided as input to a trained neural network which outputs spatially resolved flow velocities in multiple blood flow dimensions. From these outputs one could further derive and display temporal hemodynamic flow velocity profiles in the multiple blood flow dimensions at a given spatial location.

### CROSS-REFERENCE TO RELATED APPLICATIONS

This application claims priority from U.S. Provisional Patent Application 62/911,602 filed Oct. 7, 2019, which is incorporated herein by reference.

### STATEMENT OF GOVERNMENT SPONSORED SUPPORT

This invention was made with Government support under contract HD086252 awarded by the National Institutes of Health. The Government has certain rights in the invention.

### FIELD OF THE INVENTION

The present invention relates generally to estimating blood flow velocity. More particularly, the invention relates to a method of using deep learning neural networks to provide a full three-dimensional characterization of blood flow dynamics in vivo.

### BACKGROUND OF THE INVENTION

Conventional blood flow velocity estimation techniques can only measure blood flow along the transmit ultrasound beam directions. These techniques cannot measure the flow that is perpendicular to the beam directions. This produces one-dimensional (1D) velocity estimation that is inaccurate for the measurement of blood flow velocity. It also limits the application of Doppler ultrasound to blood vessels that are along with the ultrasound beam directions, or have a small angle (typically within 30 degrees) deviation from the transmit beams. In addition, due to these limitations, these techniques cannot provide a full characterization of the hemodynamics in vivo.

Efforts in obtaining two-dimensional (2D) velocity measurement of blood flow velocity involves complicated transmit or receive sequencing or beamforming, computationally intensive estimators (e.g. least-squares multiple-angle estimation) or speckle tracking. These techniques are computationally intensive and may require complex sequencing or beamforming. Efforts to alleviate these limitations may result in a compromise in image quality or equipment cost.

The present invention advances the art to provide techniques to estimate 2D blood flow velocity using deep neural networks.

### SUMMARY OF THE INVENTION

The present invention provides a method and system to characterize blood flow dynamics in vivo. A Doppler acquisition is obtained in vivo with two or more transmit-receive event pairs (step a). Each transmit event in the two or more transmit-receive event pairs at a different transmit angle, which could be two or more angles (step b). In one example the different transmit angles are three to five angles. The Doppler acquisition is processed to determine three-dimensional angle-resolved RF data. In one example, the three-dimensional angle-resolved RF data includes axial data, lateral data, and transmit angle data.

The steps (a) and (b) are then repeated to obtain four-dimensional dimensional angle-resolved RF data. In one example, the four-dimensional angle-resolved RF data includes axial data, lateral data, transmit angle data, and slow time.

The four-dimensional angle-resolved RF data is then provided as input into a trained neural network. This trained neural network is adaptive to the dimensions of the angle-resolved RF data, and outputs spatially resolved flow velocities in multiple blood flow dimensions. In one example, the multiple blood flow dimensions include at least an axial dimension and a lateral dimension.

The method and system could further include deriving from the outputs of the trained neural network and displaying temporal hemodynamic flow velocity profiles in the multiple blood flow dimensions at a given spatial location.

In one variation to plane wave transmits at different angles, the teachings of the invention could also be varied to include embodiments with transmit source and element allowing for diverging or focused transmits.

Embodiments of the invention estimate a 2D blood flow velocity using deep learning neural networks. This technique is fast and provides an accurate estimation of 2D blood flow velocity in all directions, including the flow that is perpendicular to transmit beam directions. Using 2D or other multidimensional ultrasound transducers, the technique can be improved to provide full 3D characterization of blood flow dynamics in vivo. The invention has been characterized in computer simulations, flow phantom experiments, and in vivo human liver vasculature studies.

Using existing deep learning frameworks and GPU hardware, the technique can be implemented and integrated into medical ultrasound scanners at low cost without major modification of hardware. No customized hardware is needed at all.

The invention has advantages for application that involves the measurement of blood flow velocity. For example, cardiac blood flow measurements to detect abnormal flow patterns, placental blood flow velocity measurements, tumor angiogenesis characterization, and kidney blood flow measurement, among others.

In one embodiment, a computer processor implements the training, validation, testing, and deployment of the technique.

### BRIEF DESCRIPTION OF THE DRAWINGS

FIG. 1 shows a flow chart according to an exemplary embodiment of the invention. Step 1 Doppler Acquisition: Plane wave transmits at different angles, and/or long ensemble Doppler EF data ensemble. Step 2 Receive Beamforming: Dynamic receive beamforming and/or angle resolved RF data. Step 3 Clutter Filtering: Spatiotemporal filter, IIR filter, or the like. Step 4 Velocity Estimation: Neural Network Estimator. Method 1 being Angle-Resolved RF Data, and Method 2 being Additional summation for Angle Compounded RF Data prior to Estimation. Step 5 Post-processing, visualization and quantitative analysis: Smoothing, Vector Flow Velocity Map, Hemodynamic Analysis: Resistance Index, Max/Min Velocity, etc.

FIG. 2 shows multiple angle transmits according to an exemplary embodiment of the invention.

FIG. 3 shows angle-resolved RF data as input to a neural network estimator to determine velocity estimations according to an exemplary embodiment of the invention.

FIG. 4 shows details of the neural network estimator of FIG. 3 according to an exemplary embodiment of the invention.

FIG. 5 shows a graph of velocity estimation of FIG. 3 according to an exemplary embodiment of the invention.

FIG. 6 shows according to an exemplary embodiment of the invention angle-resolved data and angle-compounded data.

FIGS. 7A-F show according to an exemplary embodiment of the invention a performance characterization. Method 1. Simulation results. Vector flow estimation results from the simulation data. (FIG. 7A) and (FIG. 7B) Two examples of vector flow velocity images. The color scale represents velocity magnitudes, and the arrows indicate flow direction. (FIG. 7C) and (FIG. 7D) Center axial cross-sections of (FIG. 7A) and (FIG. 7B), respectively. The transverse velocities are shown in gray scale (original graph in blue), and the axial velocities are shown in gray scale (original graph in red). The ground truth is shown with dashed lines and the estimated velocities are shown with solid lines. (FIG. 7E) and (FIG. 7F) Estimated peak velocities and flow angles from all 46 cases in the test set compared with ground truth. The true values are shown in x-axis and the estimated results are shown in y-axis. Each measurement is represented with one circle. The original green and yellow circles, now shown in gray scale, corresponds to the cases in (FIG. 7A) and (FIG. 7B), respectively. The original red dashed lines (now shown in gray scale) represent where the estimated values equal true values. The figures show that the technique can accurately estimate the velocity magnitude and angle.

FIGS. 8A-B show according to an exemplary embodiment of the invention a performance characterization. Method 2. Simulation results. Vector flow estimation results from the simulation data. Top row in both FIGS. 8A-B: Two examples of vector flow velocity images. The color scale represents velocity magnitudes, and the arrows indicate flow direction. Bottom row in both FIGS. 8A-B: Center axial cross-sections of the images in the top row, respectively. The transverse velocities are shown in the original graph in blue, and the axial velocities are shown in red, now both shown in gray scale. The ground truth is shown with dashed lines and the estimated velocities are shown with solid lines. The figure shows that the technique can accurately estimate the velocity magnitude and angle.

FIGS. 9A-D show according to an exemplary embodiment of the invention a performance characterization. Method 1. Flow phantom experiment results. FIG. 9A: one example flow velocity magnitude image in the flow phantom experiment. The original color scale represents velocity magnitude values, however, now shown in gray scale. FIG. 9B: center cross-sectional curves of the velocity values. The axial, lateral, and magnitude curves all indicate parabolic velocity profile, which is expected in a fully developed laminar flow. FIG. 9C (left): estimated peak velocity as a function of various volumetric flow rate. A linear relation is observed, which is as expected. FIG. 9C (right): estimated flow angles. The estimated flow angle is 108.88°, or equivalently, 18.88° to the surface of the phantom, which is close to the true value of 18°. The experimental results indicate accurate estimation of flow velocity magnitudes and angles.

FIG. 10 shows according to an exemplary embodiment of the invention human in vivo liver imaging results. Method 1. One example of flow velocity estimation in the in vivo human liver hemodynamics imaging study. Left: velocity magnitude over the entire field of view at 1972 ms. The original graph color (now in gray scale) represents velocity magnitude, and the arrows show flow directions. The arrow length is proportional to the local velocity. Right: temporal profiles of the velocity magnitude, transverse velocity, and axial velocity during the 2000 millisecond acquisition. The measurement location is indicated with the black arrow in the left image.

FIGS. 11A-E show according to an exemplary embodiment of the invention human in vivo placental imaging results. Method 1. One example of vector flow images and estimation from in vivo human placental acquisitions. (FIG. 11A) Velocity magnitude over the entire field of view at 1120 ms. For aesthetics, flow angle is not shown. (FIG. 11B) A zoomed-in vector flow image of the chorionic villi arteries. The color represents velocity magnitude, and the arrows show flow directions. The arrow length is proportional to the local velocity. (FIG. 11C) Temporal profiles of the velocity magnitude, transverse velocity, and axial velocity during the 2000 millisecond acquisition. The measurement location is indicated with the black arrow in (FIG. 11B). (FIG. 11D) Power Doppler (PD) image of the same location as (FIG. 11B), provided to show vessel location. PD has high detection sensitivity with most vessels detected but no velocity estimation. (FIG. 11E) Axial velocity estimation as would have been shown with color Doppler imaging. Note that we used a long ensemble and have higher sensitivity than clinical scanners, and that the vessels with flow that are close to transverse direction are not detected, indicated by the white arrows. For more transverse flow, the limitation would be more obvious. We can quantify the completeness of vessel detection using PD as the gold standard.

### DETAILED DESCRIPTION

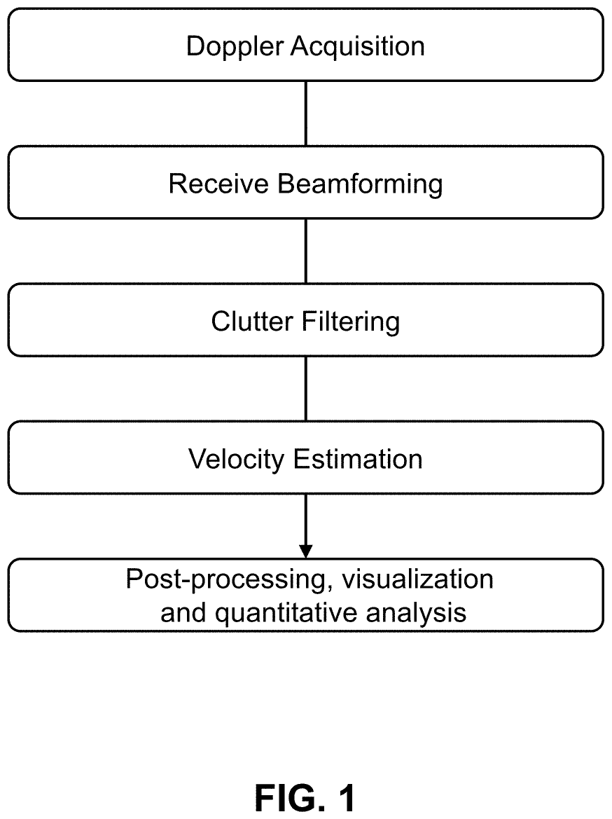

In one embodiment the invention is a method of processing signals for input to a neural network structure to output three-dimensional characterization of blood flow dynamics in vivo (FIG. 1). In this method, a Doppler signal of blood flow is acquired using an ultrasound scanner. The output data dimensions of the Doppler signal comprise 4-dimensional (4D) data. A computer processor is then used for:
i. beamforming the Doppler signal, where output data from the beamforming comprises 4D data, where the 4D data comprises axial data, azimuth data, transmit angle data, and slow time data, and where beamforming 4D data is the output.
ii. filtering the beamforming output 4D data to reshape the beamforming output 4D data into 2D data comprising axial times, azimuth times transmit event, and the slow time, where the filtering could include for example decomposition using Single Value Decomposition (SVD), energy-based thresholding or velocity-based thresholding on the singular values, SVD reconstruction. The 2D filtered data is reshaped back to the 4D data including reshaped axial data, reshaped azimuth data, reshaped transmit event data, and reshaped slow time data; and
iii. inputting the reshaped 4D data to a trained neural network to estimate a vector velocity of the blood flow from angle-resolved beam-summed RF data, or from angle-compounded beam-summed RF data.

From multiple Doppler frame acquisition data, step ((iii) could be repeated to produce videos of Doppler vector flow velocity, including magnitude and angle and producing measurement of temporal profiles of axial velocity, lateral velocity, magnitude of velocity and angle of velocity at any location in the field of view.

Embodiment of the invention are a method and system with signal processing steps as shown in FIG. 1, and list below. Examples of system include the execution of the methods steps while being integrated into a Doppler Acquisition System.

Step 1. The Doppler signal acquisition is done using the ultrasound scanner, where the output data dimensions include 4-dimensional (4D) data including:
1) axial,
2) receive channel,
3) transmit event (e.g. transmits at different angles), and
4) slow time (i.e. Doppler ensemble).

The term “slow time” is derived from Doppler radar terminology, referring to the Doppler sampling dimension. Data sampling is performed at a fixed pulse-repetition-frequency. In the invention, each sample has a 3D data cube with axial, receive channel, and transmit event dimensions. Each sample in this dimension may be referred to as one ensemble. The number of samples in this dimension may be referred to as “packet size” or “ensemble length”.

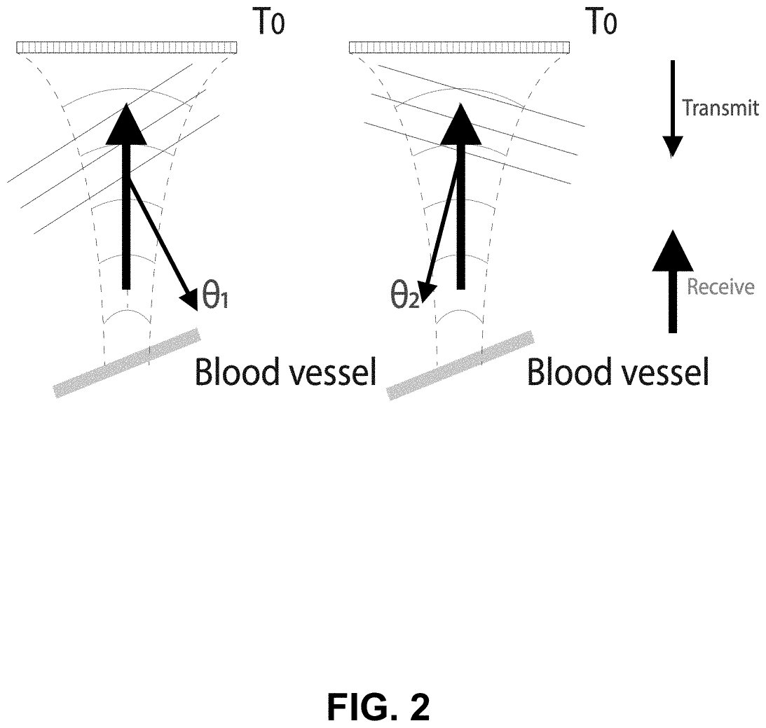

FIG. 2 shows multiple angle transmits. In this example, two plane waves are emitted consecutively at two different angles, represented by θ₁ and θ₂ respectively. The ultrasound wave backscattered by the tissue and blood cells are detected by the transducer array and are processed. In the actual acquisition, more plane wave transmits at different angles can be used in a similar way. Other types of ultrasound transducers, including phased arrays and curvilinear arrays can also be used. Other types of synthetic aperture imaging settings, including diverging wave transmits, can be used instead of the plane wave transmits.

For simplicity, “transmit events” and “transmits with different angles” are used interchangeably herein. In practice, any synthetic transmit aperture technique can be used in the transmit events, including using different diverging or focused waves, or different transmit apertures. “Angle compounding” in the following text means coherently summing data across the transmit event dimension in one Doppler acquisition. “Angle resolved” data refers to data that is not angle (or element/source) compounded.

Step 2. The Beamforming (dynamic receive beamforming) has output data dimensions: 4D data including:
1) axial,
2) azimuth (for data acquired using linear ultrasound transducers, the azimuth dimension can be interchangeably referred to as the lateral dimension),
3) transmit event (e.g. transmit angle), and
4) slow time.

Step 3. Clutter filtering can be embodied using the method of Singular Value Decomposition-based (SVD) filtering to reshape the 4D data into 2D. The reshaping vectorizes the axial, azimuth, and transmit event dimensions into one dimension, and the slow time dimension is kept as the second dimension. The resulted 2D matrix has these two dimensions:
1) axial times azimuth times transmit event, and
2) slow time.

After that, SVD decomposition, energy- or velocity-based thresholding on the singular values, SVD reconstruction are performed to remove unwanted tissue clutter and other types of noise, leaving only blood signals. The last step is reshaping the 2D filtered data back to 4D:
1) axial,
2) azimuth,
3) transmit event,
4) slow time.

Note that in the first step, the reshaping method from 4D data to 2D data is through vectorization of 3 of the 4 dimensions of data, including axial, azimuth, and transmit event dimensions, into one-dimension (1D), while preserving the fourth original dimension, that is the slow time dimension. According to further embodiments of the invention, other filters, including Butterworth filter or eigen-based filters, can be used in a similar manner as well.

In one aspect, a power Doppler image is optionally produced to show the location of blood vessel. A threshold is applied to the power Doppler image to produce a binary mask, where all pixels in blood vessel region have values of 1, and all pixels outside the blood vessel have values of 0. The mask is then multiplied to all RF data to provide additional suppression of noise outside the blood vessel.

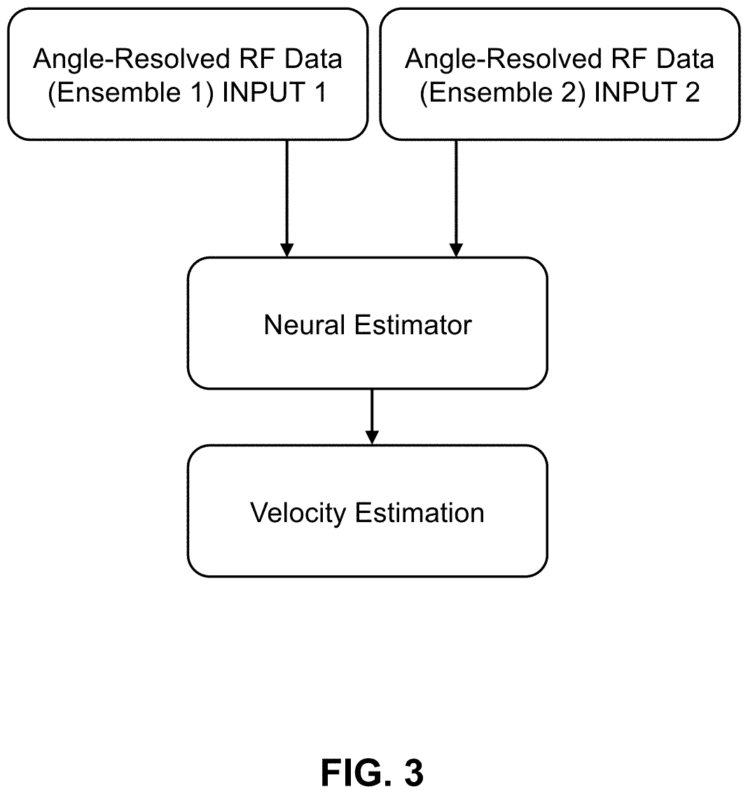

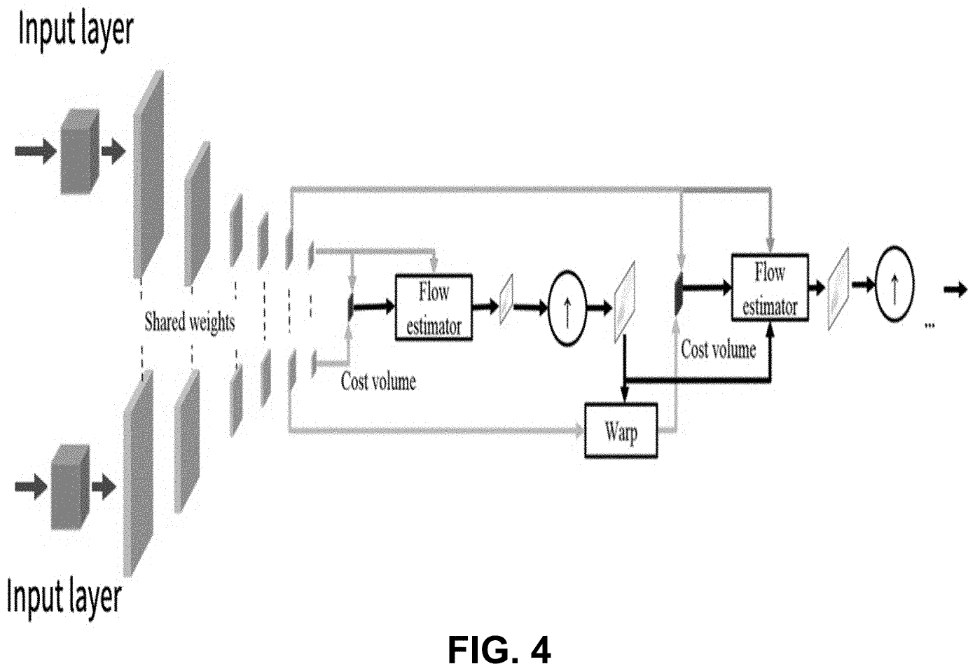

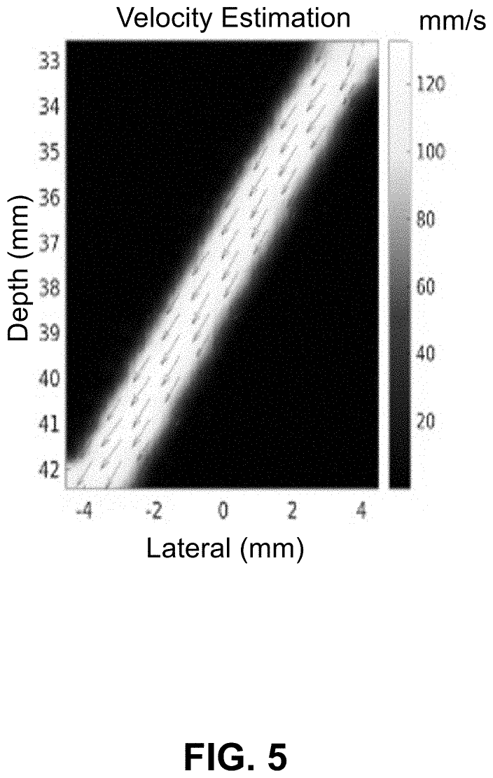

Step 4. The vector velocity estimation using neural networks includes two methods: Method 1 vector velocity estimation from angle-resolved beam-summed data using neural networks, and Method 2 vector velocity estimation from angle-compounded beam-summed data using neural networks (FIGS. 3-4).

FIGS. 3-4 shows the structure of the neural network structure, the inputs, and the outputs for method 1. The inputs are the filtered angle-resolved RF data from two consecutive Doppler acquisitions, and the output is the spatially resolved flow velocities in both axial and lateral dimensions, which is shown as equivalently the flow magnitudes and angles. The network structure has two major components: the input layer that is adaptive to the dimensions of the angle-resolved data, and the optical flow network. An optical flow network structure is shown based on the pyramid, warping, and cost volume (PWC) network. For Method 2, the input can be replaced with angle-compounded filtered RF data. In this case, the input layer has an input channel number of n=1, and the weights of the neural network can be those trained specifically for the angle-compounded method.

Method 1

The neural network used has two major components: one component is the input layer that is tailored to extract features from the angle-resolved data, and the other major component is for vector flow velocity estimation from the features. The input layer for feature extraction is one convolutional layer which input dimension is dependent upon the number of plane waves utilized in the data acquisition. For an n-angle input data, the first layer of the network needs to be tailored as a convolution layer with n input channels and 16 output channels (with kernel size being 3, stride being 1). In theory, n can be any positive integer.

Method 2

The neural network structure used in Method 2 differs from Method 1 only in the input layer. Because angle-compounded data is used as input, the input layer has n=1 input channel instead of n>1 input channels. Alternatively, the input layer can also have n>1 channels and the angle-compounded data can be copied for n times before used as the input to the network. The rest of the structure used in Method 2 is the same as Method 1. The specific weights in the network, however, may be changed adaptively in the training process. This training does not alter the structure.

Turning to Method 1, the input includes angle-resolved, beamformed beam-summed radio-frequency data acquired with two Doppler events, where the data dimensions are 4D data comprising two frames of 3D data,
1) axial,
2) azimuth,
3) transmit event (e.g. angle).

The beam-summation is performed in the receive element dimension, and is not performed in the transmit events dimension. The output includes vector flow estimation from the flow velocity field in 2D space. For each spatial location (x, z), there are two velocity estimation values corresponding to axial velocity (vz) and azimuth velocity (vx). The data dimensions are 3D, with the size of one dimension being two. The other two dimensions are azimuth and axial locations in space.

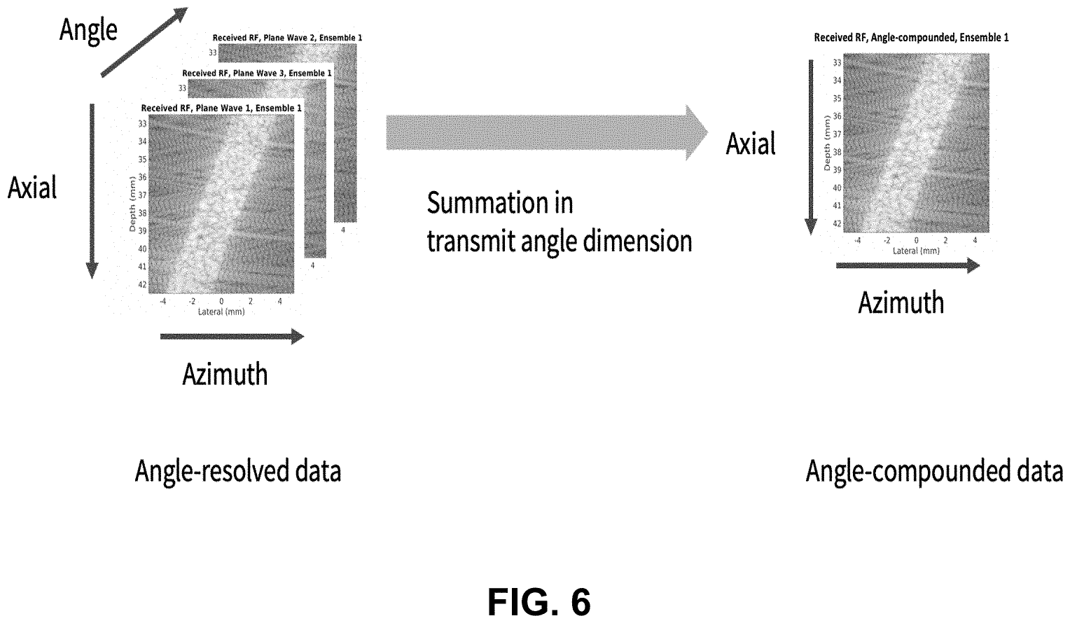

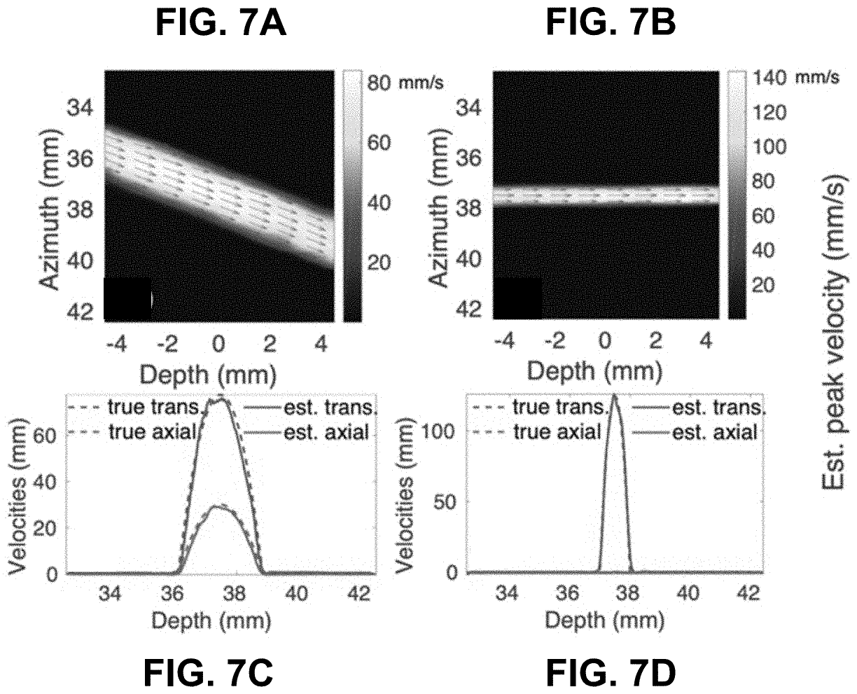

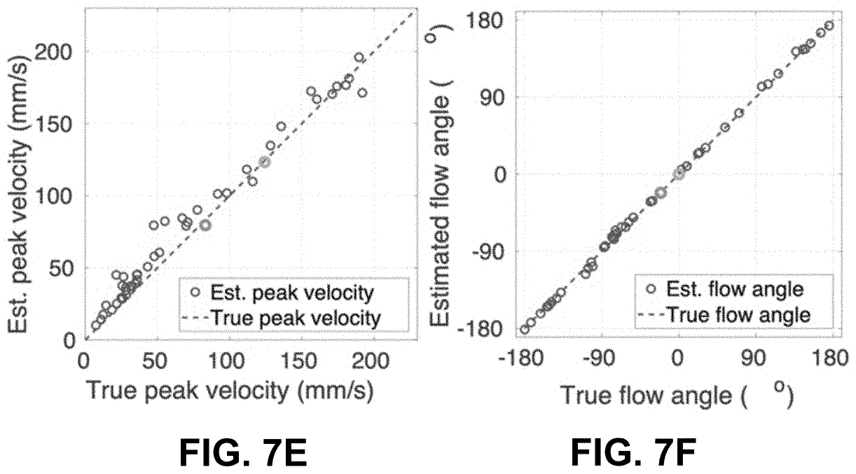

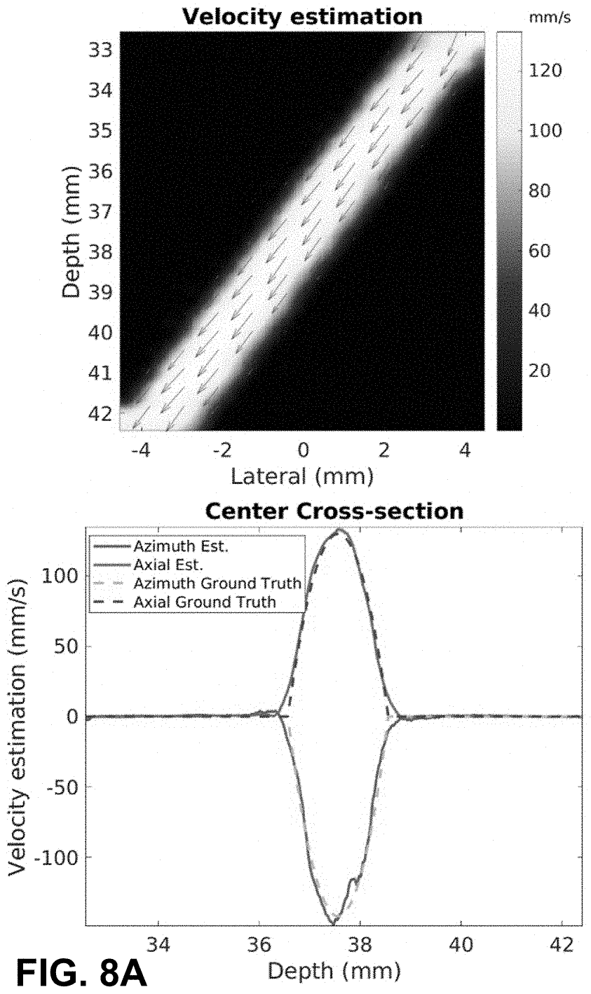

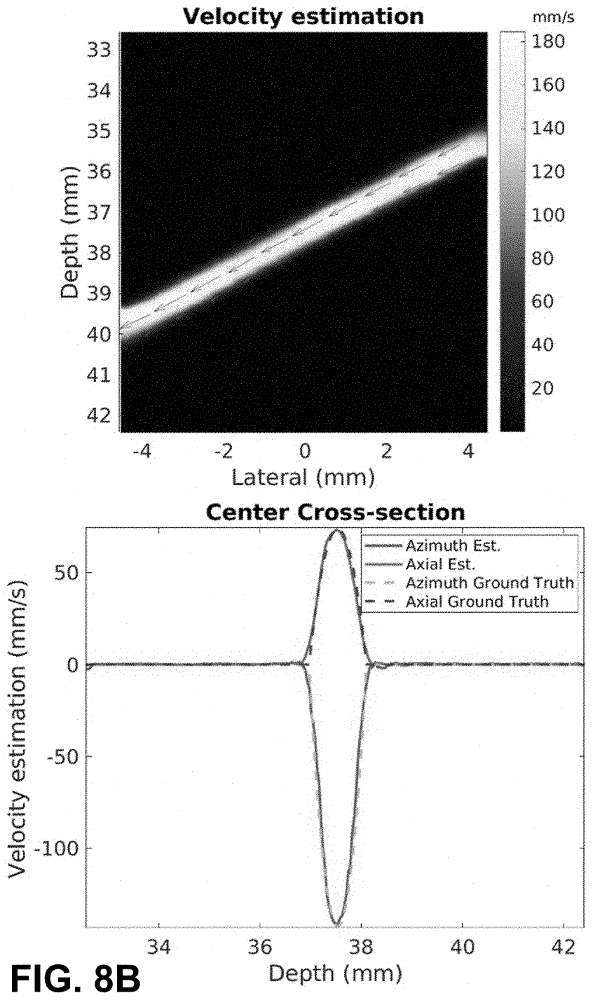

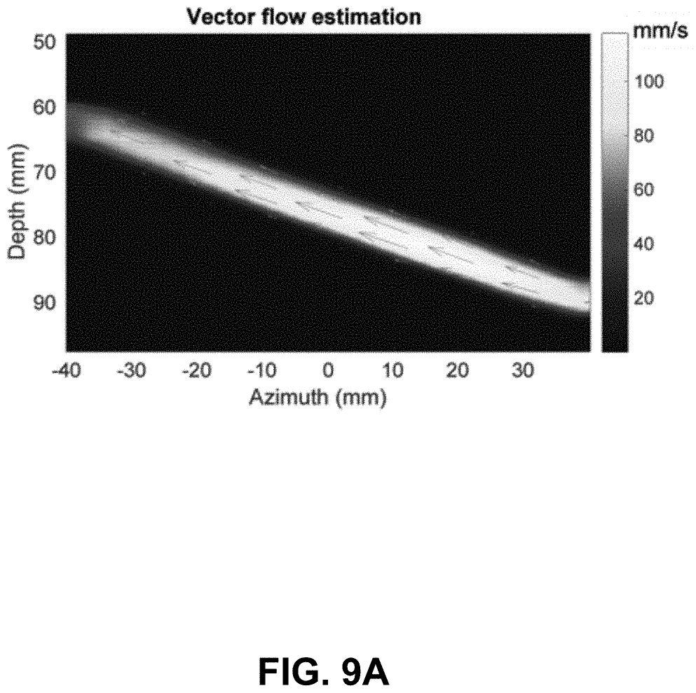

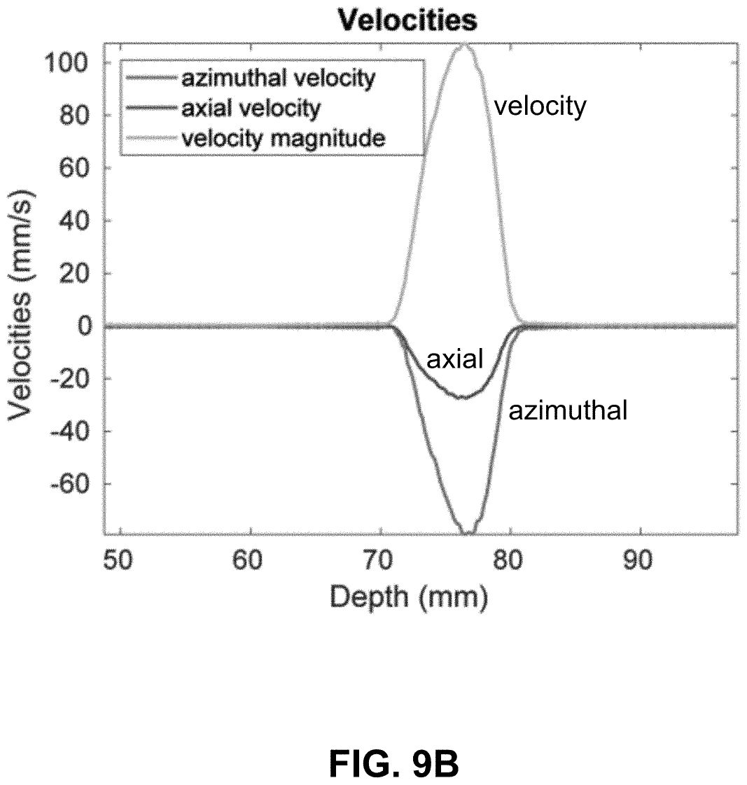

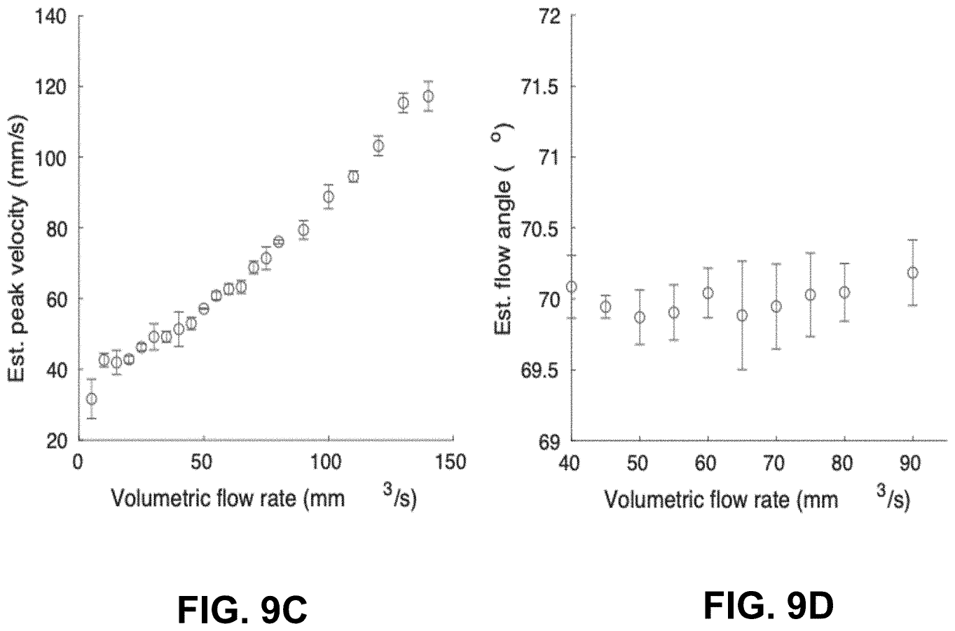

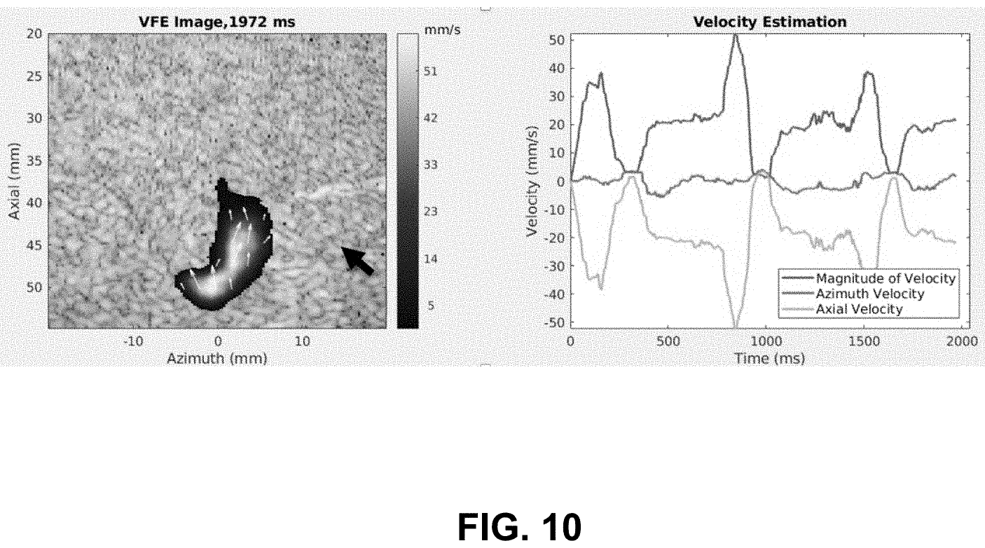

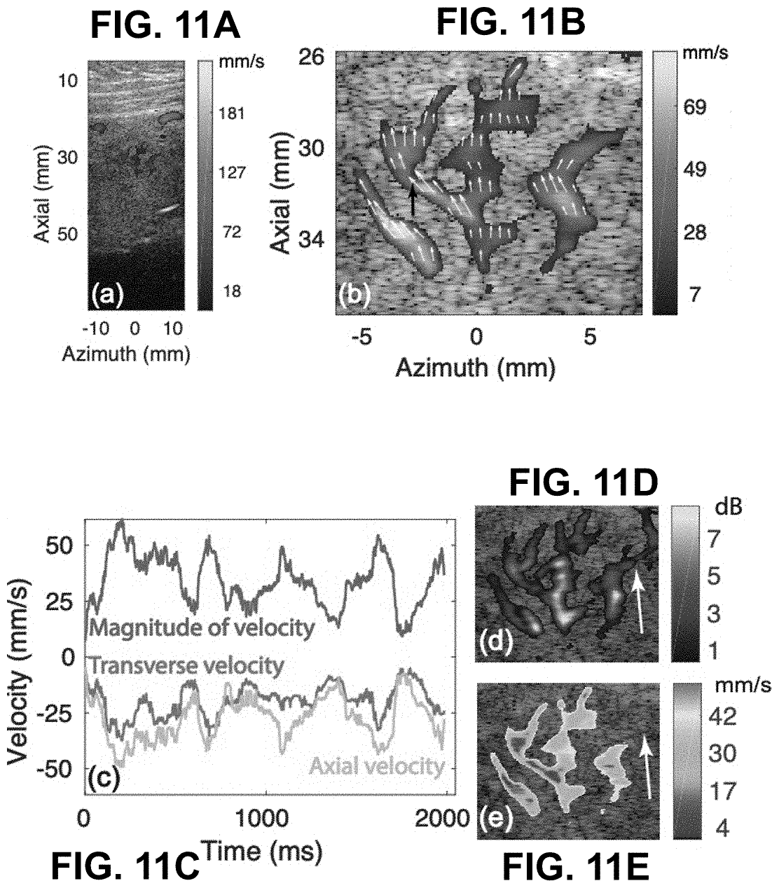

FIG. 6 shows angle-resolved data and angle-compounded data. For Method 2, an additional step is to sum the 4D angle-resolved, beamformed beam-summed radio-frequency data in the transmit event dimension, and reducing the data to 3D (azimuth, axial, slow time). FIG. 6 shows the summation of data corresponding to 1 slow time instance (labeled as Ensemble 1). The angle-resolved data for Ensemble 1 is three-dimensional: angle, axial, and azimuth. After the summation, the angle-compounded data for Ensemble 1 is two-dimensional: axial and azimuth. The process is repeated for all slow time instances, and thus converting the 4D angle-resolved data to 3D angle compounded data.

For Method 2, an additional step is performed that includes summing the 4D angle-resolved, beamformed beam-summed radio-frequency data in the transmit event dimension, and reducing the data to 3D (azimuth, axial, slow time). The data here is referred to as angle compounded beam-summed data. The input includes angle compounded beam-summed data acquired with two Doppler events, where the data dimensions include 3-dimensional data has two frames of 3D data, 1) axial, 2) azimuth. The output is a vector flow estimation, and a flow velocity field in 2D space. For each spatial location (x, z), there are two velocity estimation values corresponding to axial velocity (vz) and azimuth velocity (vx). The data dimensions include 3D, with the size of one dimension being two. The other two dimensions are azimuth and axial locations in space.

Regarding the performance difference between Method 1 and Method 2, Method 1 requires a larger data size to be used as input, because angle-resolved data is needed. The estimation is more accurate with lower variance, especially for flow in which the direction is perpendicular to transmit beam direction or ultrasound transducer axis.

Method 2 uses data with small sizes, because the angle compounded data has a smaller size than the angle resolved data from the same Doppler acquisition by a factor equal to the number of transmit events (e.g. transmit angles). It produces accurate flow estimation for flow in which the directions are not perpendicular to transmit beam direction or ultrasound transducer axis. For perpendicular flow, the variance produced using Method 2 is high than Method 1.

Returning to the signal processing steps, note that vector velocity estimation is produced from two consecutive Doppler frames (i.e. with an ensemble length of 2). For Doppler acquisitions with ensemble length greater than two, repeat Step 4 for the entire Doppler acquisition by processing all consecutive pairs of Doppler frames. The output would be the vector velocity estimation for all Doppler acquisitions. If multiple Doppler frames (more than 2 frames, preferably more than 100 for high video quality) are acquired and processed using Step 5, videos of vector flow velocity magnitudes and angles can be produced as shown in APPENDIX A of U.S. Provisional Patent Application 62/911,602 filed Oct. 7, 2019 to which this application claims the benefit and which is incorporated herein by reference. In addition, at any specific locations of the field of view (including in the blood vessels), temporal profiles of axial velocity, azimuth velocity, magnitude of velocity can be directly measured, as shown in the same APPENDIX A. The temporal profile of the angle of velocity can be calculated as the arctangent of the ratio of the axial velocity and azimuth velocity. The magnitude of the vector velocity can also be calculated. In the same APPENDIX A, the calculated angles are shown using the arrows.

$$\vert v \vert = \sqrt{{v}_{ϰ}^{2} + {v}_{z}^{2}} , \\ θ = arctan ( \frac{{v}_{z}}{{v}_{ϰ}} ) ,$$

Conversion from axial and azimuth velocities to the magnitude and angle of velocities. In this equation, νₓ is the lateral velocity, ν_{z} is the axial velocity, |ν| is the velocity magnitude, and θ is the flow angle.

Step 5 is optional, that includes performing post processing to improve the vector flow velocity estimation results. Typical methods include low-pass filtering or median filtering in spatial and/or slow time dimensions. The velocity information can be visualized as maps of flow velocities in the entire field of view. Further quantitative analysis of hemodynamics can be performed using the velocity estimation. Example of such quantitative analysis include finding the maximum or minimum of velocities, analyzing the pulse or the pulsing pattern, calculating the resistance of blood flow, etc.

Training of the neural network training includes:
i) A two-staged training process. In the first training stage, the neural network model is trained using the open access large scale optical flow datasets, including the FlyingChair dataset (https://lmb.informatik.uni-freiburg.de/resources/datasets/FlyingChairs.en.html#flyingchairs), the MPI Sintel dataset (http://sintel.is.tue.mpg.de), and the KITTI 2015 data set (http://www.cvlibs.net/datasets/kitti/eval_flow.php). In the second stage, the network with weights trained in the first stage is retrained using ultrasound flow simulation data. The rationale for the two-stage training is to utilize the large open access optical flow datasets to reduce size of the ultrasound simulation datasets, because the simulation is time consuming. training data is simulated from Doppler flow data (The simulation details are discussed in APPENDIX B of U.S. Provisional Patent Application 62/911,602 filed Oct. 7, 2019 to which this application claims the benefit and which is incorporated herein by reference. The processing details are in the system design signal processing steps discussed above).
ii) A loss function that is a summation of 5 terms:

$$L = {\sum }_{i = 1}^{5} {λ}_{i} {l}_{i}$$

λᵢ (i=1, 2, 3, 4, 5) are the weights for the 5 different losses l₁ to l₅, respectively, and can be adjusted in the training process as hyperparameters. l₁=∥predicted velocities−true velocities∥ₗ₁, in which ∥·∥ₗ₁ represents the L−1 norm. This loss calculates the L−1 norm of the difference between predicted flow velocities and the ground truth in the entire field of view.

$${l}_{2} = \frac{Total number of false zeros}{Total number of true zeros} .$$

This loss calculates the ratio between the number of false 0 velocity pixels and the number of true 0 velocity pixels.

$${l}_{3} = {‖ predicted velocities in the blood vessels - \\ true velocities in the blood vessels ‖}_{l 2}$$

n which ∥·∥ₗ₂ represents the L−2 norm. This loss calculates the L−2 norm of the difference between predicted flow velocities and the ground truth in the vessel regions.

l₄=TV(predicted axial velocities), where TV(ν) represents the total variation, defined as TV(ν)=Σ_{all pixels}|ν(z+1)−ν(z)|. It calculates the summation of the absolute values of the differences between the axial flow velocity estimations at all adjacent pixels. Minimizing this loss function is equivalent to the introduction of prior knowledge that human blood vessels are smooth and that the axial flow velocity does not have abrupt and non-continuous changes, which is based on human physiology.

l₅=TV (predicted azimuth velocities), which is the total variation of the flow estimation in the azimuth dimension. TV(ν) is defined as in l₄. Minimizing this loss function is equivalent to the introduction of prior knowledge that human blood vessels are smooth and that the lateral flow velocity does not have abrupt and non-continuous changes.

Turning now to using input data collected with multiple plane wave transmits at different angles, the Doppler flow dataset differences from the optical flow data sets are first presented, where the data in the optical flow imaging case describes motion of relatively rigid objects, including chairs, trees, cars, etc. In the videos, the objects are rigid and do have significant local deformation. The shapes are preserved relatively well from frame to frame (i.e. in slow time dimension). In addition, the three-color channels, in most cases, show objects that have highly similar shapes and describe the same motion.

The data in Doppler flow imaging is different in these two major aspects. First, the blood flow signal does not describe rigid object motion without local deformation. Instead, the signals deform in slow time dimension, and have variable rates of change in appearance that is dependent on location. For example, the signals in the center of a blood vessel move faster and change their appearances faster in slow time, compared to signals near the edge of the same blood vessel. This phenomenon is well documented and rigorously proven in the literature, and is different from the videos in the optical flow data sets. Second, the angle resolved beamformed RF data acquired using different transmit angles show different images of the same blood vessel. In other words, the appearances of the blood signals acquired using plane waves at different angles, in general, show different patterns with different textures. The difference can be measured as a decrease of the correlation between them, which has been theoretically described and experimentally demonstrated.

Turning now to the increase in the number of angles in the angle-resolved method, where first presented is the impact. The increase in the number of angles in the angle-resolved method, in general and assuming all other conditions being the same, improves the estimation accuracy by reducing the variance of estimation. The trade-off is the longer training and inference computation time, as well as a lower limit on the Doppler pulse-repetition-frequency.

According to one embodiment of the method of the invention, for an n-angle input data, the first layer of the network needs to be tailored as a convolution layer with n input channels and 16 output channels (with kernel size being 3, stride being 1).

In theory, n can be any positive integer. Specifically, the first layer can be represented as:

| Convolutional | Input | Output | Kernel | |
|---|---|---|---|---|
| Layer Number | channel number | channel number | size | Stride |
| 1 | n | 16 | 3 | 2 |

For the angle number of 5, as an example, the feature extraction pyramid network structure can be significantly different from a conventional neural network structure. The entire feature extraction pyramid network structure with angle number 5 is:

| Convolutional | Input | Output | Kernel | |
|---|---|---|---|---|
| Layer Number | channel number | channel number | size | Stride |
| 1 | 5 | 16 | 3 | 2 |
| 2 | 16 | 16 | 3 | 1 |
| 3 | 16 | 32 | 3 | 2 |
| 4 | 32 | 32 | 3 | 1 |
| 5 | 32 | 64 | 3 | 2 |
| 6 | 64 | 64 | 3 | 1 |
| 7 | 64 | 96 | 3 | 2 |
| 8 | 96 | 96 | 3 | 1 |
| 8 | 96 | 128 | 3 | 2 |
| 10 | 128 | 128 | 3 | 1 |
| 11 | 128 | 196 | 3 | 2 |
| 12 | 196 | 196 | 3 | 1 |

The training with the network with this feature pyramid achieves fast convergence within 2 epochs.

What is claimed is:

1. A method to characterize blood flow dynamics in vivo, comprising:
(a) obtaining a Doppler acquisition in vivo with two or more transmit-receive event pairs, with each transmit event in the two or more transmit-receive event pairs at a different transmit angle;
(b) processing the Doppler acquisition to determine three-dimensional angle-resolved RF data;
(c) repeating the steps (a) and (b) to obtain four-dimensional dimensional angle-resolved RF data; and
(d) inputting the four-dimensional angle-resolved RF data into a trained neural network, wherein the trained neural network is adaptive to the dimensions of the angle-resolved RF data, and wherein the trained neural network outputs spatially resolved flow velocities in multiple blood flow dimensions.

2) The method as set forth in claim 1, wherein the different transmit angles are two or more angles.

3) The method as set forth in claim 1, wherein the different transmit angles are three to five angles.

4) The method as set forth in claim 1, wherein the three-dimensional angle-resolved RF data comprises axial data, lateral data, and transmit angle data.

5) The method as set forth in claim 1, wherein the four-dimensional angle-resolved RF data comprises axial data, lateral data, transmit angle data, and slow time.

6) The method as set forth in claim 1, wherein the multiple blood flow dimensions comprise at least an axial dimension and a lateral dimension.

7) The method as set forth in claim 1, further comprising deriving from the outputs of the trained neural network and displaying temporal hemodynamic flow velocity profiles in the multiple blood flow dimensions at a given spatial location.
